(19) United States
(12) Patent Application Publication
(10) Pub. No.: US 2004/0180812 A1
(43) Pub. Date: Sep. 16, 2004

(54) Methods of treating and preventing proliferative disease

(75) Inventors: Adam P. Dicker, Cherry Hill, NJ (US); Randy Burd, Philadelphia, PA (US); Kulbir Sidhu, Philadelphia, PA (US)

Correspondence Address: David S. Resnick, NIXON PEABODY LLP, 100 Summer Street, Boston, MA, 02110, US

(73) Assignee: Technology Center

(21) Appl. No.: 10/737,360

(22) Filed: Dec. 15, 2003

Related U.S. Application Data
(60) Provisional application No. 60/433,471, filed on Dec. 13, 2002.

(51) Int. Cl. A61K 38/14; A61K 31/7076; A61K 31/7072; A61K 31/7048; A61K 31/704; A61K 31/522; A61K 31/513

(52) U.S. Cl. 514/8; 514/27; 514/109; 514/47; 514/50; 514/263.31; 514/269; 514/34; 514/283; 514/410; 514/251; 514/182; 514/449; 600/1

(57) ABSTRACT

The present invention provides methods of treating proliferative disease in a patient (e.g., a mammal such as a human) in need of such treatment, said treatment comprising administering, concurrently or sequentially, an effective amount of (1) an anti-platelet or anti-clotting agent and (2) an anti-neoplastic agent and/or radiation therapy. A second method of treatment comprises administering Plavix, also known as clopidogrel, or SR 25909 to a patient in need of such treatment. An additional method comprises administering an anti-platelet or anti-clotting agent to an individual at risk for developing proliferative disease. The methods of the present invention are particularly useful for the treatment or prevention of various cancers, especially epithelial cancers, e.g., prostate cancer, lung cancer, breast cancer, colorectal cancer, and pancreatic cancer. In preferred embodiments, the anti-platelet agent is combined with one of the following antineoplastic agents: taxotere, gemcitabine, paclitaxel (Taxol®), 5-Fluorouracil (5-FU), cyclophosphamide (Cytoxan®), temozolomide, or Vincristine.

This Application claims the benefit of U.S. Provisional Application No. 60/433,471, filed Dec. 13, 2002.

### FIELD OF THE INVENTION

The present application is directed to a method of treating subjects afflicted with proliferative diseases, such as cancers, tumors, or metastatic disease. In particular, this invention provides methods for cancer therapy, improving the therapeutic ratio for ionizing radiation and chemo-radiation, and cancer prevention. More specifically, the present invention is based, in part, on the use of pharmaceutical compositions containing therapeutically effective amounts of an anti-platelet or anti-clotting agent and/or an anti-neoplastic agent and/or radiation therapy for treating a solid tumor.

### BACKGROUND OF THE INVENTION

The treatment of cancer has thus far proved problematic. While “cancers” share many characteristics, each particular cancer has its own specific characteristics. Genetics and environmental factors have a complex interplay in the severity and prognosis of treatment. Thus, treatment must be carefully tailored.

Therapy for cancer has largely involved the use of surgery, chemotherapy, in which highly toxic chemicals are given to the patient, and/or by radiotherapy, in which toxic doses of radiation are directed at the patient. However, results with these measures, while beneficial in some tumors, has had only marginal or no effect in many others. Furthermore, these approaches often have unacceptable toxicity. While commonly effective to kill huge numbers of cancer cells, these “cytotoxic” treatments also kill extraordinary numbers of healthy cells, causing the patient to experience acute debilitating symptoms including nausea, diarrhea, hypersensitivity to light, hair loss, etc. The side effects of these cytotoxic compounds limits the frequency and dosage at which they can be administered. Such disabling side effects can be mitigated to some degree by using compounds that selectively target cycling cells, i.e., interfering with DNA replication or other growth processes in cells that are actively reproducing. Since cancer cells are characterized by their extraordinary ability to proliferate, such protocols preferentially kill a larger proportion of cancer cells in comparison to healthy cells, but cytotoxicity and ancillary sickness remains a problem.

Although cancer chemotherapy has advanced dramatically in recent years, treating cancers with a single agent has had limited success. First, any single agent may only target a subset of the total population of malignant cells present, leaving a subpopulation of cancerous cells to continue growing. Second, cells develop resistance upon prolonged exposure to a drug. Combination therapies, which employ two or more agents with differing mechanisms of action and differing toxicities, have been useful for circumventing drug resistance and increasing the target cell population. In addition, certain combinations of agents may be synergistic: their combined effect is larger than that predicted based on their individual activities. Thus, combining different agents can be a powerful strategy for treating cancer. However, combination therapies are a hit or miss proposition. In many cases, cross effects and treatment load can result in lower effectiveness for the combination than either treatment alone. Muiltidrug resistance can also be a problem.

With only a few exceptions, no single drug or drug combination is curative for most cancers. Thus, new drugs or combinations that can prolong the onset of life-threatening tumors and/or improve quality of life by further reducing tumor load are very important.

Failure of anti-cancer agents to reach all clonogenic cells at cytotoxic concentrations is recognized as an important form of resistance to solid tumors (Simpson-Herren, L., et al., 1991, Cell Prolif. 24: 355-65; Tunggal, J. et al., 1999, Clin. Cancer Res. 5: 1583-6). This has led to the development of sensitizing agents, such as Taxotere. Taxotere is a cytotoxic chemotherapy that is frequently used as a radiosensitizer. Radiosensitization is achieved through re-oxygenation of hypoxic cells, G2/M cell cycle arrest and a decrease in interstitial fluid pressure (Griffon-Etienne, G. et al., 1999, Cancer Res. 59: 3776-82; Milas, L. et al., 1999, Semin. Radiat. Oncol. 9: 12-26; Milas, L. et al., 1995, Cancer Res. 55: 3564-8). Compromise of the vascular system through vessel thrombosis during fractionated radiotherapy may also limit the penetration of Taxotere when used concurrently with or following radiation.

Thus a new treatment regiment that can improve the therapeutic ratio for ionizing radiation and chemotherapy is needed for improved, more effective cancer treatment.

There are a number of individuals at increased risk for cancer based on genetics, environment, or lifestyle. There are few treatments available that are safe and effective at reducing this risk with relatively minor side effects. Thus, new drugs or combinations that can decrease the risk of proliferative disease in at risk individuals would be of great benefit.

### SUMMARY OF THE INVENTION

The present invention provides methods of treating proliferative disease in a patient (e.g., a mammal such as a human) in need of such treatment, said treatment comprising administering, concurrently or sequentially, an effective amount of (1) an anti-platelet or anti-clotting agent and (2) an anti-neoplastic agent and/or radiation therapy. The present invention also provides a method of treatment comprises administering Plavix, also known as clopidogrel, or SR 25909 to a patient in need of such treatment. The present invention further provides a method comprises administering an anti-platelet or anti-clotting agent to an individual at risk for developing proliferative disease. The methods of the present invention are particularly useful for the treatment or prevention of various cancers, especially epithelial cancers, e.g., prostate cancer, lung cancer, breast cancer, colorectal cancer, and pancreatic cancer. In preferred embodiments, the anti-platelet agent is combined with one of the following antineoplastic agents: taxotere, gemcitabine, paclitaxel (Taxol®), 5-Fluorouracil (5-FU), cyclophosphamide (Cytoxan®), temozolomide, or Vincristine.

In a preferred embodiment, the present invention provides a method of treating cancer, comprising administering, concurrently or sequentially, an effective amount of (1) an anti-platelet agent, such as Plavix, and (2) Taxotere.

### BRIEF DESCRIPTION OF THE DRAWINGS

FIGS. 1A and B show U-87 cell proliferation in the presence of clopidogrel (FIG. 1A) and Aggrenox™ (FIG. 1B) in three concentrations. Measured using Coulter counter. Results are the mean±s.e.m. of three independent experiments.

FIGS. 2A and B show clonogenic survival of U-87 and A-549 cells treated with Aggrenox™ (FIG. 2A) and clopidogrel (FIG. 2B). Cells were exposed to three concentrations of each drug. Colonies were counted and the mean±s.e.m. is shown.

FIG. 3 shows tumor growth rate in vivo. U-87 cells were inoculated into the right hind limb of athymic NCR NUM mice. Clopidogrel was added to the drinking water in concentrations of 0.135 (low), 0.180 (drug level 2) or 0.225 mg/ml (drug level 1). A non-solubilized version (original drug) of clopidogrel was also used in one group.

FIG. 4 shows tumor growth with Plavix treatment. Panels A-C describe regression plots of percent tumor growth for each treatment. Days are number of days post 60 mm³ tumor volume. Panel D describes the relation of each treatment group. A 5-day growth delay is recorded for the 60 mg/kg/day treatment.

FIG. 5 shows tumor growth with Plavix and Radiation Treatment. Panels A-D describe regression plots of tumor growth in mm³ for each treatment. The X-axis describes the number of days post first recorded tumor volume. Tumors were allowed to reach 60 mm³ before Plavix treatment and 100 mm3 before X-ray irradiation. Arrows indicate point of X-ray treatment. Data does not include outliers.

FIG. 6 shows regression plots of U-87 Xenograph with Plavix and Radiation Treatment. The graph describes the relation of each treatment group. Vertical lines indicate growth delay between treatment groups.

### DETAILED DESCRIPTION OF THE INVENTION

We have now discovered methods of treating and preventing proliferative diseases, especially cancers, by (1) combining anti-platelet or anti-clotting agents with a method for treating cancer (using a chemotherapeutic agent and/or radiation therapy); (2) administering Plavix, also known as clopidogrel, or SR 25909 to a patient in need of such treatment; (3) administering an anti-platelet or anti-clotting agent to an individual at risk for developing proliferative disease.

The method of treating proliferative diseases, according to this invention, includes a method for treating (inhibiting) the abnormal growth of cells, including transformed cells, in a patient in need of such treatment (e.g., a mammal such as a human), by administering, concurrently or sequentially, an effective amount of an anti-platelet or anti-clotting agent and an effective amount of a chemotherapeutic agent and/or radiation. Abnormal growth of cells means cell growth independent of normal regulatory mechanisms (e.g., loss of contact inhibition), including the abnormal growth of benign and malignant cells of other proliferative diseases.

In preferred embodiments, the methods of the present invention include methods for treating or inhibiting tumor growth in a patient in need of such treatment (e.g., a mammal such as a human) by (1) administering, concurrently or sequentially, an effective amount of an anti-platelet or anti-clotting agent and an effective amount of an antineoplastic agent and/or radiation therapy; (2) administering Plavix or SR 25909; or (3) administering an anti-platelet or anti-clotting agent to an at-risk individual. Malignant and metastatic conditions which can be treated or prevented with methods of the present invention include, but are not limited to, the solid tumors listed in Table 1 (for a review of such disorders, see Fishman et al., 1985, Medicine, 2d Ed., J. B. Lippincott Co., Philadelphia) and blood-borne tumors such as leukemias.

TABLE 1
MALIGNANCIES AND RELATED DISORDERS

Solid tumors

sarcomas and carcinomas

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms’ tumor
cervical cancer
testicular tumor
lung carcinoma
small cell lung carcinoma
bladder carcinoma
epithelial carcinoma
glioma
astrocytoma
medulloblastoma
craniopharyngioma
ependymoma
Kaposi's sarcoma
pinealoma
hemangioblastoma
acoustic neuroma
oligodendroglioma
menangioma
melanoma
neuroblastoma
retinoblastoma

As used herein the following terms have the following meanings unless indicated otherwise:

antineoplastic agent—a chemotherapeutic agent effective against cancer;

concurrently—(1) simultaneously in time, or (2) at different times during the course of a common treatment schedule; and

sequentially—(1) administration of one component of the method ((a) Anti-platelet agent, or (b) antineoplastic agent and/or radiation therapy) followed by administration of the other component; after administration of one component, the second component can be administered substantially immediately after the first component, or the second component can be administered after an effective time period after the first component; the effective time period is the amount of time given for realization of maximum benefit from the administration of the first component.

Demonstration of Therapeutic or Prophylactic Utility

The Therapeutics of the invention can be tested in vivo for the desired therapeutic or prophylactic activity as well as for determination of therapeutically effective dosage. For example, such compounds can be tested in suitable animal model systems prior to testing in humans, including, but not limited to, rats, mice, chicken, cows, monkeys, rabbits, etc. For in vivo testing, prior to administration to humans, any animal model system known in the art may be used.

Chemotherapeutic Agents

Classes of compounds that can be used as the chemotherapeutic agent (antineoplastic agent) include: alkylating agents, antimetabolites, natural products and their derivatives, hormones and steroids (including synthetic analogs), and synthetics. Examples of compounds within these classes are given below.

Alkylating agents (including nitrogen mustards, ethylenimine derivatives, alkyl sulfonates, nitrosoureas and triazenes): Uracil mustard, Chlormethine, Cyclophosphamide (Cytoxan.™), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (including folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (including vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, paclitaxel (paclitaxel is commercially available as Taxol® and is described in more detail below in the subsection entitled “Microtubule Affecting Agents”), Mithramycin, Deoxycoformycin, Mitomycin-C, L-Asparaginase, Interferons (especially IFN-a), Etoposide, and Teniposide.

Hormones and steroids (including synthetic analogs): 17.alpha.-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Zoladex.

Synthetics (including inorganic complexes such as platinum coordination complexes): Cisplatin, Carboplatin, Hydroxyurea, Amsacrine, Procarbazine, Mitotane, Mitoxantrone, Levamisole; and Hexamethylmelamine.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 2002 edition (Medical Economics Company, Montvale, N.J. 07645-1742, USA); the disclosure of which is incorporated herein by reference thereto.

Microtuble Affecting Agents

As explained above, the present invention also provides methods of treating diseased cells by contacting the cells with an anti-platelet agent and a microtubule affecting agent (e.g., paclitaxel, a paclitaxel derivative or a paclitaxel-like compound). As used herein, a microtubule affecting agent is a compound that interferes with cellular mitosis, i.e., having an anti-mitotic effect, by affecting microtubule formation and/or action. Such agents can be; for instance, microtubule stabilizing agents or agents which disrupt microtubule formation.

Microtubule affecting agents useful in the invention are well known to those of skill in the art and include, but are not limited to allocoichicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol.™ derivatives (e.g., derivatives (e.g., NSC 608832), Taxotere, thiocolchicine (NSC 361792), trityl cysteine (NSC 83265), vinblastine sulfate (NSC 49842), vincristine sulfate (NSC 67574), epothilone A, epothilone, and discodermolide (see Service, (1996) Science, 274:2009) estramustine, nocodazole, MAP4, and the like. Examples of such agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

Particularly preferred agents are compounds with paclitaxel-like activity. These include, but are not limited to paclitaxel and paclitaxel derivatives (paclitaxel-like compounds) and analogues. Paclitaxel and its derivatives are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Generally, activity of a test compound is determined by contacting a cell with that compound and determining whether or not the cell cycle is disrupted, in particular, through the inhibition of a mitotic event. Such inhibition may be mediated by disruption of the mitotic apparatus, e.g., disruption of normal spindle formation. Cells in which mitosis is interrupted may be characterized by altered morphology (e.g., microtubule compaction, increased chromosome number, etc.).

In a preferred embodiment, compounds with possible tubulin polymerization activity are screened in vitro. In a preferred embodiment, the compounds are screened against cultured WR21 cells (derived from line 69-2 wap-ras mice) for inhibition of proliferation and/or for altered cellular morphology, in particular for microtubule compaction. In vivo screening of positive-testing compounds can then be performed using nude mice bearing the WR21 tumor cells. Detailed protocols for this screening method are described by Porter (1995) Lab. Anim. Sci., 45(2):145-150.

Other methods of screening compounds for desired activity are well known to those of skill in the art. Typically such assays involve assays for inhibition of microtubule assembly and/or disassembly. Assays for microtubule assembly are described, for example, by Gaskin et al. (1974) J. Molec. Biol., 89: 737-758. U.S. Pat. No. 5,569,720 also provides in vitro and in vivo assays for compounds with paclitaxel-like activity.

Methods for the safe and effective administration of the above-mentioned microtubule affecting agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

Anti-Platelet and Anti-Clotting Agents

Compromised blood vessels resulting from a course of fractionated radiotherapy cause decreased penetration of chemotherapeutic and radiosensitizing agents, such as Taxotere. The compromise is caused by platelet aggregation and can be minimized by the use of an anti-platelet or anti-clotting agent. This intervention will result in increased penetration of the chemotherapeutic and radiosensitizing agents during and after radiotherapy. Compounds that can be used as anti-platelet and anti-clotting agents include: Plavix, Abciximab, Reopro, Fondaparinux Sodium, Arixtra, Argatroban, Novastan, Streptokinase, Streptase, Ticlopidine, Ticlid, Reteplase, Retavase, Alteplase, Activase, Tenecteplase, TNKase, Eptifibatide, Integrilin, Tinzaparin, Innohep, Lepirudin, Refludan, Dalteparin, Fragmin, Dipyridamole, Aggrenox, Antithrombin III Human, Thrombate 3, Anagrelide, Agrylin, Cilostazol, Pletal, Tirofiban, Aggrastat, Pentoxifyline, Trental, Warfarin, Coumadin, Danaparoid, Orgaran, Bivalirudin, Angiomax, Fondaparinux, Organon, Ancrod, Viprinex, Epoprostenol, Flolan, Cangrelor, or Ximelagatran.

An additional compound, SR 25909, is also included as an effective treatment.

Pharmaceutical Compositions

Inert, pharmaceutically acceptable carriers used for preparing pharmaceutical compositions of the anti-platelet agents and the chemotherapeutic agents described herein can be either solid or liquid. Solid preparations include powders, tablets, dispersible granules, capsules, cachets and suppositories. The powders and tablets may comprise from about 5 to about 70% active ingredient. Suitable solid carriers are known in the art, e.g., magnesium carbonate, magnesium stearate, talc, sugar, and/or lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into conveniently sized molds, allowed to cool and thereby solidify.

Liquid preparations include solutions, suspensions and emulsions. As an example may be mentioned water or water-propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The anti-platelet agents and the chemotherapeutic agents described herein may also be deliverable transdermally. The transdermal compositions can take the form of creams, lotions, aerosols and/or emulsions and can be included in a transdermal patch of the matrix or reservoir type as are conventional in the art for this purpose.

The suitability of a particular route of administration will depend on the pharmaceutical compositions (e.g., whether they can be administered orally without decomposing prior to entering the blood stream) and the disease being treated. For example, treatment of solid tumors on the skin or on exposed mucosal tissue may be more effective if the pharmaceutical compositions are administered topically, transdermally, or mucosally (e.g., by nasal, sublingual, buccal, rectal, or vaginal administration). Treatment of solid tumors within the body may be more effective if the pharmaceutical compositions are administered parenterally or orally. Similarly, parenteral administration may be preferred for the acute treatment of a solid tumor, whereas transdermal or subcutaneous routes of administration may be employed for chronic treatment or prevention of a solid tumor.

In a preferred form, the Therapeutic is administered in combination with a subcutaneously-implanted biodegradable, biocompatible polymeric implant or pump which releases the troponin subunit, fragment or homolog thereof over a controlled period of time at a selected site. Examples of preferred polymeric materials include polyanhydrides, polyorthoesters, polyglycolic acid, polylactic acid, polyethylene vinyl acetate, and copolymers and blends thereof. See, Medical Applications of Controlled Release, Langer and Wise (eds.), 1974, CRC Pres., Boca Raton, Fla.; Controlled Drug Bioavailability, Drug Product Design and Performance, Smolen and Ball (eds.), 1984, Wiley, New York; Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105. In yet another embodiment, a controlled release system can be placed in proximity of the therapeutic target, i.e., the brain, thus requiring only a fraction of the systemic dose (see, e.g., Goodson, in Medical Applications of Controlled Release, 1989, supra, vol. 2, pp. 115-138).

Preferably the compounds are administered orally.

Preferably, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the anti-platelet agents and the chemotherapeutic agents and/or radiation therapy will be regulated according to the judgment of the attending clinician (physician) considering such factors as age, condition and size of the patient as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

In the methods of this invention, an anti-platelet or anti-clotting agent is administered concurrently or sequentially with a chemotherapeutic agent and/or radiation. Thus, it is not necessary that, for example, the chemotherapeutic agent and the anti-platelet agent, or the radiation and the anti-platelet agent, should be administered simultaneously or essentially simultaneously. The advantage of a simultaneous or essentially simultaneous administration is well within the determination of the skilled clinician.

Also, in general, the anti-platelet or anti-clotting agent and the chemotherapeutic agent do not have to be administered in the same pharmaceutical composition, and may, because of different physical and chemical characteristics, have to be administered by different routes. For example, the anti-platelet or anti-clotting agent may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of anti-platelet or anti-clotting agent, and chemotherapeutic agent and/or radiation will depend upon the diagnosis of the attending physicians and their judgement of the condition of the patient and the appropriate treatment protocol.

The anti-platelet or anti-clotting agent, and chemotherapeutic agent and/or radiation may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the anti-platelet or anti-clotting agent.

If the anti-platelet or anti-clotting agent, and the chemotherapeutic agent and/or radiation are not administered simultaneously or essentially simultaneously, then the initial order of administration of the anti-platelet or anti-clotting agent, and the chemotherapeutic agent and/or radiation, may not be important. Thus, the anti-platelet or anti-clotting agent may be administered first followed by the administration of the chemotherapeutic agent and/or radiation; or the chemotherapeutic agent and/or radiation may be administered first followed by the administration of the anti-platelet or anti-clotting agent. This alternate administration may be repeated during a single treatment protocol. The determination of the order of administration, and the number of repititions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of the anti-platelet or anti-clotting agent followed, where determined advantageous, by the administration of the chemotherapeutic agent and/or radiation, and so on until the treatment protocol is complete.

Thus, in accordance with experience and knowledge, the practising physician can modify each protocol for the administration of a component (therapeutic agent—i.e., anti-platelet or anti-clotting agent, chemotherapeutic agent or radiation) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radio-logical studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### EXAMPLE 1

### Materials and Methods

U-87 glioblastoma cells and A-549 non-small cell lung cancer cells (American Type Culture Collection) were maintained in alpha MEM (Sigma) with 10% fetal bovine serum (Atlanta Biologicals). Tumor cell lines were expanded as a monolayer culture by serial passage on tissue culture plates in tumor media (alpha MEM with 10% fetal bovine serum).

Vertebrate Animals:

The present study used ICR nu/nu mice obtained from Taconic Farms, Inc. (Germantown, N.Y.). Males of 5-8 wk of age were used. All animals were housed in microisolator cages (4/cage) within the Central Animal Facility and our laboratory laminar flow animal rooms This Facility is A.A.A.L.A.C. certified, meets all standards prescribed by the “Guide for the Care and Use of Laboratory Animals” (ISBN 0-309-05377-3, Published by the Natl. Acad. Press, Wash. D.C., revised 1998). All mice used for experimentation were anesthetized with Ketamine/Acepromazine or sodium pentobarbital anesthesia. Euthanasia was accomplished by inhalation of CO₂.

In vivo—Tumor Growth in Mice

In order to evaluate the effect of clopidogrel on U-87 tumor growth rate, U-87 cell suspension was injected subcutaneously into the right hind limb (5×10⁵ cells in 50 μl PBS) of athymic NCR NUM mice (Taconic Farms). The clopidogrel was added to the drinking water in concentrations of 0.135 (low), 0.180 (intermediate) or 0.225 mg/ml (high). This was equivalent to an oral dose of approximately 15, 20, or 25 mg/kg/day. Mice were not pretreated prior to tumor implantation. The mice were randomized to five groups (control, low, intermediate and high concentration and non-solubilized drug) with five mice per group.

U-87 tumors were allowed to grow for approximately 10-14 days until reaching an approximate volume of 40-85 mm^{3,} before treatment. Tumor volume was determined daily by direct measurement with calipers and calculated by the formula [(smallest diameter²×widest diameter)/2]. Daily weights were obtained.

In-vitro Determination of Proliferation

The cell doubling time was determined for both U-87 and A-549 tumor cells. A total of 1×10⁶ Cells from resting culture were seeded in 50 mm flasks with medium and allowed to adhere for approximately Three hours. Clopidogrel and Aggrenox™ were then added in concentrations of 25, 50, or 100 μM. After further incubation for four hours, controls and treated dishes were trypsinized and counted in a Coulter Counter ZM. This count served as the starting count for proliferation and confirmed similar cell adhesion in each series. Cells were then counted every 24 hours for a total of 96 hours.

Clonogenic Assay

The effect of clopidogrel and Aggrenox™ on U-87 and A-549 survival was determined by preparing single cell suspensions from each cell culture. The cells were subjected to three concentrations of each drug (25, 50, or 100 μM) for four hours in medium. The suspensions were serially diluted with medium. The cells were then seeded in triplicate in 50 mm flasks. After 6 to 14 days of incubation, the cultures were stained with crystal violet and counted.

Statistical Analysis.

Polynomial regression lines were fit to the data for each individual mouse for 20-24 days. Means were taken at two-day intervals for mice in each group.

Results

Inhibition of Cell Proliferation by Clopidogrel and Aggrenox™

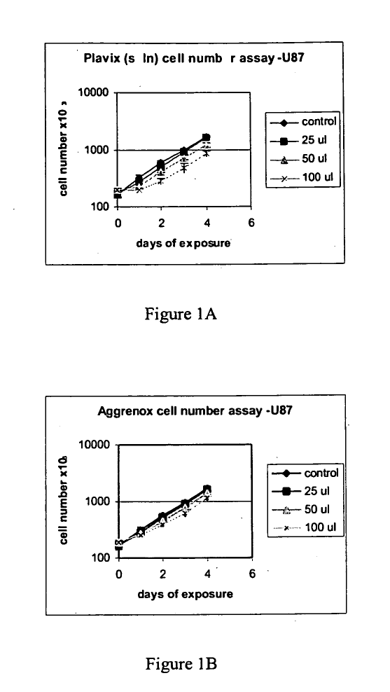

Proliferation of U-87 and A-549 cells was analysed in the presence of three concentrations of clopidogrel and Aggrenox™ using the coulter counter. The highest concentration of clopidogrel (100 μM) considerably inhibited the proliferation of U-87 cells and even more drastically inhibited A-549 cell proliferation. The same concentration of Aggrenox™ also showed less activity in both cell lines than clopidogrel. The A-549 cell line was again more sensitive to the drug than the U-87. The lowest concentrations of each drug did not significantly affect cell proliferation (FIG. 1).

Effect of Clopidogrel and Aggrenox™ on Cell Survival

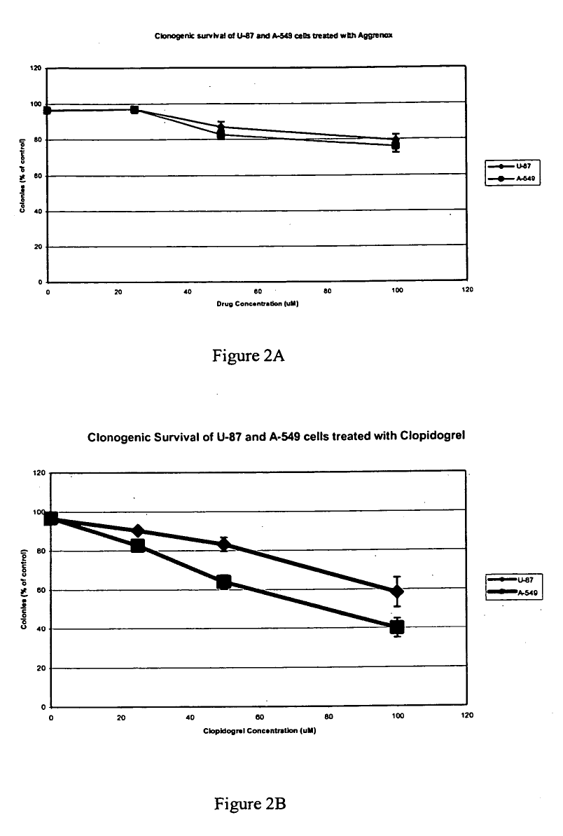

The clonogenic survival of U-87 and A-549 cells was analysed after exposure for four hours to three concentrations (25, 50, 100 μM) of clopidogrel and Aggrenox™. In the two lowest concentrations Aggrenox™ did not decrease U-87 cell survival and A-549 cells were only minimally effected. The highest concentration showed more pronounced activity. These results were exaggerated when clopidogrel was used (FIG. 2).

In-vivo Tumor Growth Delay

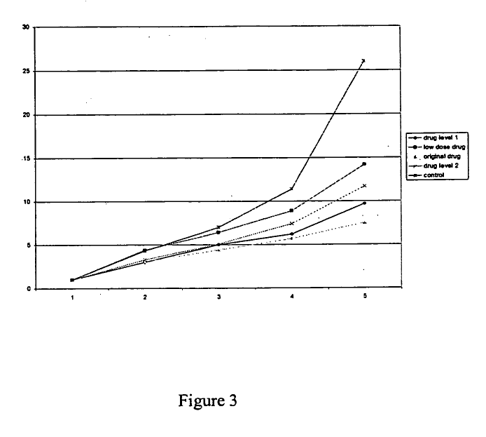

U-87 cells were inoculated into the right hind limb of athymic NCR NUM mice. Primary tumors developed equally in control and treatment group mice. U-87 tumors were allowed to grow for approximately 10-14 days until reaching an approximate volume of 40-85 mm³, before treatment. Clopidogrel was added to the drinking water in concentrations of 0.135 (low), 0.180 (intermediate) or 0.225 mg/ml (high). This was equivalent to an oral dose of approximately 15, 20, or 25 mg/kg/day. Tumor dimensions and animal weights were taken daily. A small growth delay was measured for the lowest concentration of drug. The growth delay was much more dramatic for the highest concentration of drug. A dose response was noted (FIG. 3).

### EXAMPLE 2

Without wishing to be bound by theory, we believe that interfering with platelet function as an anti-cancer agent-will be of benefit due to: (1) unique role expressed in neoplasia relative to normal tissues in terms of structure, amount, location, timing, and/or activity; (2) functionally important in initiating or promoting cancer development and progression; (3) accessible to applied interventions; (4) amenable to manipulation and (5) when regulated artificially regulated will result in measurable and reliable clinical benefits (e.g., analgesia, reduced rates of lesion incidence, recurrence, grade; improved quality of life, etc.).

Four complementary lines of evidence will substantiate antiplatelet agents as important agents for cancer prevention and as adjuncts to treatment: (1) antiplatelet agents (inhibitors) stimulate anti-cancer effects in in vitro systems; (2) antiplatelet agents and antiplatelet agents gene deletions—inhibit carcinogenesis in carcinogen-induced and genetically-driven rodent models; (3) antiplatelet agents will reduce the incidence of precancerous lesions (e.g., adenomas); cancer incidence; and cancer mortality and (4) antiplatelet agents will regress precancerous lesions (i.e., colorectal aberrant crypt foci and adenomas, and actinic keratoses of the skin) in genetic and sporadic cancer risk cohorts. We anticipate that the protective and therapeutic effects of antiplatelet agents in a broad range of tissues (e.g., skin, oral mucosa, esophagus, head and neck, gynecologic, breast, colorectal, CNS, GU, lung, bladder, myeloma, acute myelogenous and chronic lymphocytic leukemias etc.) as well.

Although these agents are relatively safe, there are some potential toxicities. These toxicities may have greater significance in the context of long-term drug exposure. At least two strategies may improve the therapeutic index of anti-platelet agents used with chemopreventive or therapeutic intent.

1) The therapeutic index of anti-platelet agents may be improved by topical applications that concentrate the anti-platelet agents on the intended target while minimizing systemic side effects. For example, Wattenberg recently demonstrated the improved efficacy and reduced toxicity of inhalational, as opposed to oral, corticosteroids (e.g., dexamethasone, budesonide) in a carcinogen-induced model of lung neoplasia (1-3). In a similar vein, topical COX inhibitors are being tested as anti-neoplastic agents in several NCI-sponsored clinical trials.

2) The optimal dose of antiplatelet agents for chemoprevention has not been determined. It may be that for chemopreventive use a lower dose is sufficient compared to therapeutic intent

We envision that the anti-cancer properties of antiplatelet agents will follow a similar development cycle to the inhibitors of COX-2. As the specific molecular mechanisms that account for the anti-cancer effects of antiplatelet agents are understood, this will facilitate the identification of agents with the most favorable therapeutic indices from among this class of compounds. While challenging, opportunities to identify new agents with greater mechanistic specificity abound. Based on impressive pre-clinical efficacy data, COX-2 selective inhibitors advanced into clinical cancer prevention and therapy trials. We anticipate similar trials testing the efficacy of anti-platelet and anti-clotting agents.

Our initial animal data supports the in vivo activity of clopidogrel alone in tumor regression. We anticipate similar results using other antiplatelet and anti-clotting drugs. In the future, antiplatelet/anticlotting drugs may be administered at the initial diagnosis of malignancy or upon failure of other treatment regimens. We expect further therapeutic benefit when these agents are combined with radiotherapy and anti-cancer drugs. We expect to find an additive and/or synergistic improvement in efficacy when anti-platelet/anti-clotting drugs are administered either concurrently, sequentially or adjuvantly. Although the mechanism of such is not currently clear, there could be a possible anti-angiogenic effect or a mechanical effect whereby the drug therapy is able to penetrate deeper into the tumor because the physical presence of clots formed by platelet aggregation have been removed.

Antiplatelet/Anticlotting drugs will have a role in the treatment of cancer and in the reduction of the incidence of cancer in patients who are deemed to have a higher risk of developing a cancer. This may include patients that have a known genetic predisposition or a medical condition, occupation, or environment that puts them at higher risk of developing a cancer.

### EXAMPLE 3

### Treatment of Human U87 Gliobastoma Tumor Xenografts with Plavix

Methods

Tissue Culture

Glioblastoma cell line U-87 (ATCC) was cultured in 150 cm² polystyrene flasks (Fisher cat. no. 355001) in Alpha MEM media (Cellgro® cat. no. 15-012-CV) supplemented with 10% FBS, 1× non essential amino acids (Cellgro® cat. no. 25-025-Cl), 0.1% glucose (Sigma® cat. no. G8644), 2 mM L-glutamine (Gibco BRL® cat. no. 25030-081) and 0.2% sodium bicarbonate (Cellgro® cat. no. 25-035-Cl) to sub-confluence (80%). Prior to cell dissociation, the culture was washed once with 1×PBS (Bio Whittaker® cat. no. 16-006Y) and dissociated with trypsin free Cellstripper™ (Cellgro® cat. no. 25-056-Cl). Suspended cells were counted using the model Z1 Coulter® particle counter.

Xenographs

Male mice (NCR-NU-M, Taconic®), 3-5 weeks old, were anesthetized with 0.2 mls Nembutal® (10 mg/ml in sterile water) subcutaneous in the back of the neck. Once anesthetized, each mouse was injected subcutaneous with 5×10⁵ cells/0.2 ml in the right hind flank. Tumors were allowed to grow to 60 mm³ prior to treatment. Tumor volume was calculated by the following formula from orthogonal measurements using Fisher Scientific® electronic digital calipers.

$$Tumor Volume = \frac{l \times {w}^{2}}{2}$$

Tumors were allowed to grow to a final volume of 2000 mm³ where upon the treatment was terminated and the mice were sacrificed using a CO2 gas chamber.

Plavix® Treatment

Treatment groups were evaluated for tumor growth delay. A 60 mg/kg/day solution of Plavix® (clopidogrel bisulfate) was prepared by crushing and dissolving 75 mg tablets in water (pH 1.0). The solute was filtered through a Nalgene® filtration unit (cat. no. 126-0020) and the pH raised to 1.78. Mice were gavaged daily with 0.2 ml solution of Plavix® and vehicle controls were gavaged daily with 0.2 ml acidified water pH 1.78.

Ionizing Radiation

Prior to X-ray irradiation, mice were anesthetized with 75 g/kg Ketamine HCl (Ketaset III®, Fort Dodge®) and 0.35 g/kg Acepromazine Maleate (Phoenix pharmaceutical, Inc.) by subcutaneous injection in the back to the neck. Tumors on the right hind leg were exposed to 7.5 Gy three times on alternate days for one week using a Pantek X-ray irradiator.

Platelet Aggregation

Mice were sacrificed with CO2 gas and immediately processed for blood collection. A volume of 0.9 ml blood was drawn from the heart with a 1 cc syringe containing 0.1 ml of 3.2% sodium citrate. To obtain at least 1.0 ml of blood, mouse blood samples were pooled (n=3). The pooled blood sample was spun at 100×g for 15 min. The supernatant (platelet rich plasma, PRP) was collected in a 1.5 ml microfuge tube and half was transferred to a new 1.5 ml tube for preparation of platelet poor plasma (PPP). The PPP tube was spun at 1000×g for 15 min and the supernatant (PPP fraction) was collected in a 1.5 ml microfuge tube. PRP and PPP fractions were diluted 1:1 with saline (37° C.). Platelet aggregation was detected by turbidometric technique in the presence of thrombin (1.0 units/ml, Chrono-log® cat. no. 386) using a Chrono-log® Whole Blood Aggregometer. Specifically, 50 μl of thrombin reagent was added to 500 μl of either PPP (blank) or PRP.

Results

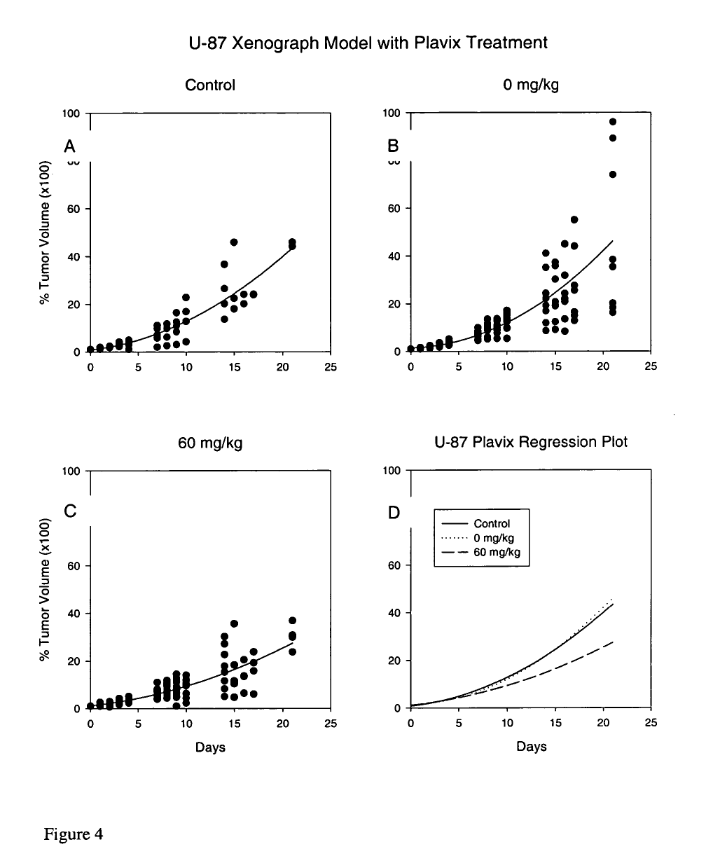

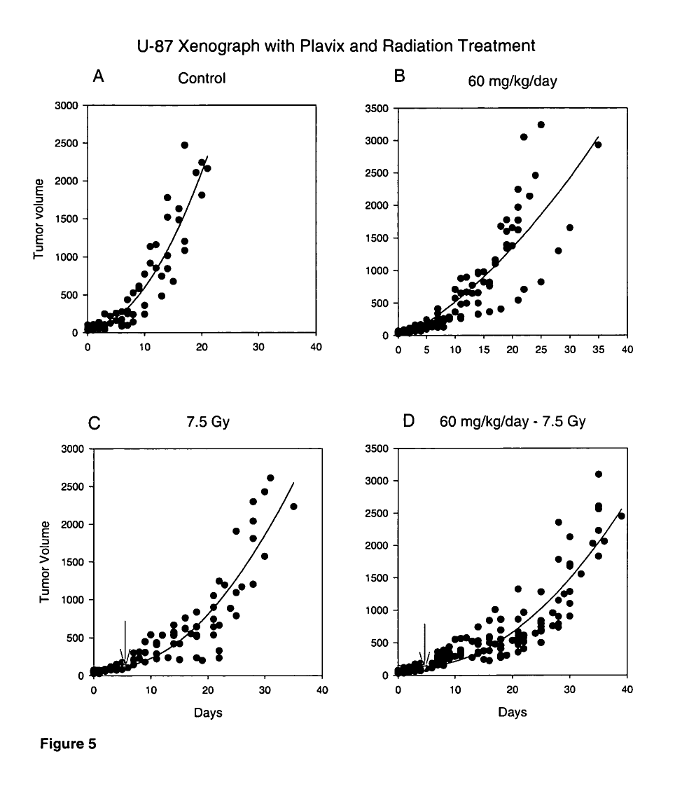

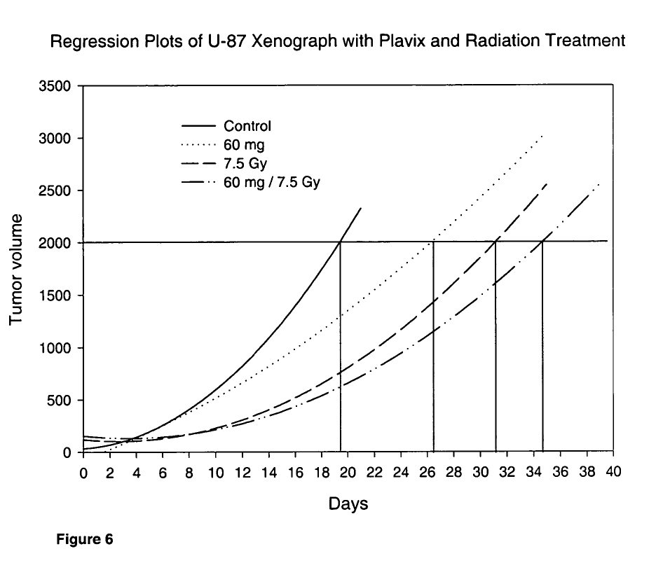

Treatment of human U87 gliobastoma tumor xenografts with Plavix alone resulted in a 5 day tumor growth delay (TGD). Treatment of the tumors with radiation increased the TGD to 12 days, while treatment with radiation and Plavix combined increased the TGD to 16 days (4 days more than radiation alone). Platelet aggregation was also significantly reduced in the Plavix treated mice indicating that the drug was biologically active in the treated mice. Therefore, treatment with Plavix alone can reduce the rate of tumor growth and increase the effectiveness of radiation. The results are illustrated in FIGS. 4-6 and a summary of tumor growth delay data is found in Table 1.

TABLE 1
Summary of Tumor Growth Delay Data.

| | | | Radiation plus |
|---|---|---|---|
| Treatment | Plavix | Radiation | Plavix |
| Tumor growth | 5 days | | |
| Delay (Experiment 1) | | | |
| Tumor growth | 5 days | 12 days | 16 days |
| Delay (Experiment | | | |
| 2) | | | |

Platelet Aggregation

To determine the anti platelet aggregation activity of Plavix® for the 60 mg/kg/day treatment, we tested the aggregation response of platelets stimulated with thrombin. Mice with 60 mg/kg/day treatment exhibited no detectable platelet aggregation compared to control mice (data not shown). This is consistent with the putative action of Plavix® as an inhibitor of adenosine diphophate (ADP)-induced platelet aggregation by direct inhibition of ADP binding to the P2Y12 receptor.

All references described herein are incorporated herein by reference.

References

1. Estensen, R. D. and Wattenberg, L. W., Studies of chemopreventive effects of myo-inositol on benzo[a]pyrene-induced neoplasia of the lung and forestomach of female A/J mice. Carcinogenesis 14: 1975-7; 1993.

2. Wattenberg, L. W., and Estensen, R. D., Chemopreventive effects of myo-inositol and dexamethasone on benzo[a]pyrene and 4-(methylnitrosoamino)-1-(3-pyridyl)-1-butanone-induced pulmonary carcinogenesis in female A/J mice. Cancer Res. 56: 5132-5; 1996.

3. Wattenberg, L. W., et al., Chemoprevention of pulmonary carcinogenesis by aerosolized budesonide in female A/J mice. Cancer Res. 57: 5489-92; 1997.

We claim:

1. A method of treating a proliferative disease in a patient in need of such treatment, said treatment comprising administering, concurrently or sequentially, an effective amount of (1) an anti-platelet agent and (2) an anti-neoplastic agent and/or radiation therapy.

2. A method of treating a proliferative disease in a patient in need of such treatment, said treatment comprising administering an effective amount of Plavix or SR 25909.

3. A method of inhibiting or preventing proliferative disease in a patient at increased risk by administering an effective amount of an anti-platelet or anti-clotting agent or SR 25909.

4. The method of claim 1, further comprising administering a radiosensitizing agent.

5. The method of claim 1 wherein said anti-platelet agent and antineoplastic agent and/or radiation are administered concurrently.

6. The method of claim 1 wherein said anti-platelet agent and antineoplastic agent and/or radiation are administered simultaneously.

7. The method of claim 1 wherein said anti-platelet agent and antineoplastic agent and/or radiation are administered sequentially.

8. The method of claim 1 wherein said antineoplastic agent and/or radiation therapy is administered first.

9. The method of claim 1 wherein said anti-platelet agent is administered first.

10. The method of claims 1-3 wherein said proliferative disease is selected from the diseases set forth in Table 1.

11. The method of claim 1 wherein said antineoplastic agent is selected from the group consisting of Uracil mustard, Chlormethine, Clyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Temozolomide, Methotrexate, 5-Fluorouracil, Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Paclitaxel, Mithramycin, Deoxycoformycin, Mitomycin-C, L-Asparaginase, Interferons, Etoposide, Teniposide 17-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estrmustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, CPT-11, Anastrazole, Letrazole, Capecitabine, Reloxafine, Droloxafine, or Hexamethylmelamine.

12. The method of claim 1 wherein said radiation is ionizing radiation.

13. The method of claims 1-3 wherein the anti-platelet or anti-clotting agent is Plavix, Abciximab, Reopro, Fondaparinux Sodium, Arixtra, Argatroban, Novastan, Streptokinase, Streptase, Ticlopidine, Ticlid, Reteplase, Retavase, Alteplase, Activase, Tenecteplase, TNKase, Eptifibatide, Integrilin, Tinzaparin, Innohep, Lepirudin, Refludan, Dalteparin, Fragmin, Dipyridamole, Aggrenox, Antithrombin III Human, Thrombate 3, Anagrelide, Agrylin, Cilostazol, Pletal, Tirofiban, Aggrastat, Pentoxifyline, Trental, Warfarin, Coumadin, Danaparoid, Orgaran, Bivalirudin, Angiomax, Fondaparinux, Organon, Ancrod, Viprinex, Epoprostenol, Flolan, Cangrelor, or Ximelagatran.

14. The method of claim 13 wherein the preferred anti-platelet agent is Plavix.

15. The method of claim 4 wherein the radiosensitizing agent is Taxotere.

16. A method of treating a proliferative disease in a patient in need of such treatment, said treatment comprising administering, concurrently or sequentially, an effective amount of (1) Plavix and (2) radiation therapy in conjunction with Taxotere.
